(19) United States
(12) Patent Application Publication
(10) Pub. No.: US 2008/0319226 A1
(43) Pub. Date: Dec. 25, 2008

(54) Process For the Preparation of Highly Pure (E) N,N-Diethyl-2-Cyano-3-(3,4-Dihydroxy-5-Nitro Phenyl) Acrylamide (Entacapone)

(75) Inventors: Venkatasubramanian Radhakrishnan Tarur, Mumbai (IN); Dhananjay Govind Sathe, Thane (IN); Nandu Baban Bhise, Mumbai (IN); Avinash Venkatraman Naidu, Thane (IN); Umesh Parashram Aher, Kalyan (IN); Sachin Shivaji Patil, Kalyan (IN)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: USV LIMITED, Govandi, Mumbai 400 080, Maharashtra (IN)

(21) Appl. No.: 12/092,657

(22) PCT Filed: Nov. 9, 2005
(86) PCT No.: PCT/IN05/00363

§ 371 Date: Aug. 18, 2008

(51) Int. Cl. C07C 231/00

(52) U.S. Cl. 564/124

(57) ABSTRACT

A process for the preparation of highly pure (E)-N,N-diethyl-2-cyano-3-(3,4-dihydroxy-5-nitrophenyl)acrylamide (Entacapone). It comprises condensing 3,4-dihydroxy-5-nitro benzaldehyde with N,N-diethyl cyano acetamide in an organic solvent in the presence of an organic base and an acid under reflux conditions followed by distilling off the solvent under vacuum and treating the residue with a lower aliphatic carboxylic acid at 40-8O0 C. The resulting residue is extracted with a chlorinated solvent and the solvent is distilled off under vacuum. The resulting residue is extracted with an organic solvent to obtain crude N,N-diethyl-2-cyano-3-3,4-dihydroxy-5-nitrophenyl)acrylamide. The crude product N,N-diethyl-2-cyano-3-(3,4-dihydroxy-5-nitrophenyl) acrylamide formed is treated with a mixture of organic alcohol and organic acid in the molar ratio of the crude product to organic alcohol 1:5 to 15 and crude product to organic acid 1:1 to 3 under reflux conditions.

### TECHNICAL FIELD

The invention relates to a process for the preparation of highly pure (E) N,N-diethyl-2-cyano-3-(3,4-dihydroxy-5-nitro phenyl)acrylamide (Entacapone) of the formula I:

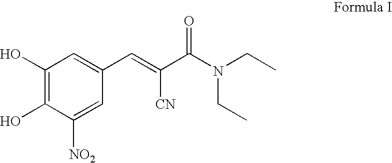

### BACKGROUND AND PRIOR ART

U.S. Pat. No. 4,963,590 describes catechol derivatives including N,N-diethyl-2-cyano-3-(3,4-dihydroxy-5-nitro phenyl)acrylamide as being useful in the treatment of diseases like central or peripheral nervous system disorders, Parkinson's disease or parkinsonian disorders. Process for the preparation of N,N-diethyl-2-cyano-3-(3,4-dihydroxy-5-nitro phenyl)acrylamide comprises condensation of 3,4-dihydroxy-5-nitro benzaldehyde and N,N-hydrochloric diethyl cyanoacetamide in the presence of piperidine-acetate in dry ethanol (Example 100). The condensation reaction and yield are reported to be very long (overnight) and 73%, respectively. The process is not, therefore, commercially viable for industrial scale production of entacapone and economical.

U.S. Pat. No. 5,135,950 reports that N,N-diethyl-2-cyano-3-(3,4-dihydroxy-5-nitrophenyl)acrylamide is a mixture of two geometric isomers namely E- and Z-isomers (70-80% E-isomer and 30-20% Z-isomer) of the following formulae I and II, respectively:

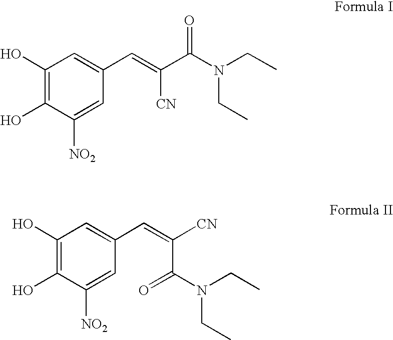

This patent relates to a stable and crystallographically essentially pure polymorphic form A of E-isomer of N,N-diethyl-2-cyano-3-(3,4-dihydroxy-5-nitrophenyl)acrylamide obtained by purifying the crude product by recrystallisation from lower aliphatic carboxylic acid such as formic acid or acetic acid with catalytic amount of hydrochloric or hydrobromic acid. The purification procedure increases the process duration and renders it commercially unviable for industrial scale production.

WO 2005/063693 describes a process for the preparation of entacapone using 3-alkoxy-4-hydroxy-5-nitrobenzaldehyde.

WO 2005/070881 describes a process for the production of (E)-entacapone polymorphic form A. 3,4-Dihydroxy-5-nitrobenzaldehyde is condensed with N,N-diethylcyanoacetamide in the presence of a base like piperidine in alcohol like isopropanol. The reaction mixture is poured into a mixture of aqueous ethyl acetate and water followed by pH adjustment with acetic acid. The product is separated from the organic layer to yield (E) isomer of entacapone having 99.7% purity. The condensation reaction itself is reported to be quite long (10-15 hours).

Novel polymorphic forms C and D of entacapone are disclosed in WO2005/066117. 3,4-Dihydroxy-5-nitrobenzaldehyde is condensed with N,N-diethylcyanoacetamide in the presence of a base like piperidine in isopropanol followed by addition of acetic acid and crystallization with a mixture of alcohols to obtain polymorphic form C. Polymorphic form D is obtained from form C or form A by dissolution in solvents such as polar organic solvents aliphatic alcohols or sulfoxides.

WO 2005/063695 and WO 2005/063696 also disclose novel polymorphs C, D and E of Entacapone by crystallisation.

### OBJECTS OF THE INVENTION

An object of the invention is to provide a process for the preparation of (E)-N,N-diethyl-2-cyano-3-(3,4-dihydroxy-5-nitrophenyl)acrylamide, which gives the product in high purity and good yield.

Another object of the invention is to provide a process for the preparation of (E)-N,N-diethyl-2-cyano-3-(3,4-dihydroxy-5-nitrophenyl)acrylamide, which reduces the process duration and is simple, easy and convenient to carry out and is commercially viable and economical.

Another object of the invention is to provide (E)-N,N-diethyl-2-cyano-3-(3,4-dihydroxy-5-nitrophenyl)acrylamide in very high purity and good yield.

### DETAILED DESCRIPTION OF INVENTION

According to the invention there is provided a process for the preparation of highly pure (E)-N,N-diethyl-2-cyano-3-(3,4-dihydroxy-5-nitrophenyl)acrylamide (Entacapone) of the formula I

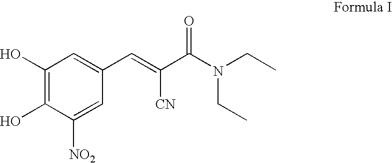

comprising
i) condensing 3,4-dihydroxy-5-nitro benzaldehyde with N,N-diethyl cyano acetamide in an organic solvent in the presence of an organic base and an acid under reflux conditions followed by distilling off the solvent under vacuum and treating the residue with a lower aliphatic carboxylic acid at 40-80° C.;
ii) extracting the residue with a chlorinated solvent and distilling off the solvent under vacuum;
iii) extracting the residue with an organic solvent to obtain crude N,N-diethyl-2-cyano-3-3,4-dihydroxy-5-nitrophenyl)acrylamide; and
iv) treating the crude product N,N-diethyl-2-cyano-3-(3,4-dihydroxy-5-nitrophenyl)acrylamide with a mixture of organic alcohol and organic acid in the molar ratio of the crude product to organic alcohol 1:5 to 15 and crude product to organic acid 1:1 to 3 under reflux conditions.

The organic solvent used in step (i) is selected from lower aliphatic alcohol like methanol, ethanol, n-propanol, isopropanol or mixtures thereof or is preferably ethanol. The organic base used in step (i) is selected from piperidine morpholine, N-methyl morpholine or pyrrolidine or is preferably piperidine. The acid used in step (i) is organic acid selected from lower aliphatic carboxylic acid or inorganic acid selected from sulfuric acid or hydrochloric acid. The lower aliphatic carboxylic acid used in step (i) is preferably acetic acid or formic acid. Preferably, the residue in step (i) is treated at 65° C.

The chlorinated solvent used in step (ii) is selected from methylene chloride or chloroform or is preferably methylene chloride.

The organic solvent used in step (iii) is selected from polar and non-polar solvents such as alcohols, ketones, esters or mixtures thereof. The organic solvent is preferably ester selected from methyl acetate, ethyl acetate, propyl acetate or mixtures thereof or is preferably ethyl acetate.

The mixture of organic alcohol and organic acid used in step (iv) is selected from aliphatic alcohol like methanol, ethanol, n-propanol or isopropanol and lower aliphatic carboxylic acid like acetic acid, formic acid, methane sulfonic acid or trifluoro acetic acid or is preferably, methanol and acetic acid.

Preferably, the molar ratio of the crude product to organic alcohol is 1:8 and the crude product to organic acid is 1:2.

The product namely N,N-diethyl-2-cyano-3-(3,4-dihydroxy-5-nitrophenyl)acrylamide is recovered from the reaction mixture of step (iv) by filtering the reaction mixture, precipitating the product from the filtrate by gradually cooling the filtrate at 0-30° C. preferably 10-50° C. and drying the precipitate at 60-70° C. preferably in an electric oven.

The process of the invention affords (E)-N,N-diethyl-2-cyano-3-(3,4-dihydroxy-5-nitrophenyl)acrylamide in high purity (>99.8%) and in good yield (about 53.68%) with Z-isomer content <0.5%. The process duration is reduced and the process is simple, easy and convenient to carry out and is commercially viable and economical.

The following experimental example is illustrative of the invention but not limitative of the scope thereof:

### EXAMPLE 1

A mixture of 3,4-dihydroxy-5-nitro benzaldehyde (500 g), N,N-diethyl cyano acetamide (575 ml), acetic acid (375 ml) and piperidine (500 ml) in ethanol (4.51) were refluxed for 6 hrs. Ethanol was distilled off under vacuum and 1.5 l of formic acid was added to the residue at 65° C. and stirred for 30 mins at 65° C. The reaction mixture was cooled to R T and charged with methylene dichloride. The organic layer was separated and washed twice with 2×3.5 l water. Methylene dichloride was distilled off under vacuum from the organic layer and the residue was treated with ethyl acetate 1.25 l. The yellow solid was filtered out from the solvent, washed with 1.25 l ethyl acetate and dried in an electric oven at 60-70° C. to obtain crude N,N-diethyl-2-cyano-3-(3,4-dihydroxy-5-nitro phenyl)acrylamide.

Yield of crude entacapone 495 g (59.63%), Purity 99.2++

Z-isomer 0.5%

The crude product (495 g), methanol (3960 ml) and acetic acid (990 ml) were refluxed for 1 hr. The clear solution was filtered and the product was precipitated by gradually cooling the filtrate to 10-15° C. The solid was filtered out, washed twice with cold ethyl acetate (2×990 ml) at 15° C._and dried in an electric oven at 60-70° C.

Yield of entacapone based on the crude product 445.5 g (90%). Purity 99.8%.

Z-isomer 0.02%

1. A process for the preparation of highly pure (E)-N,N-diethyl-2-cyano-3-(3,4-dihydroxy-5-nitrophenyl)acrylamide (Entacapone) of the formula I 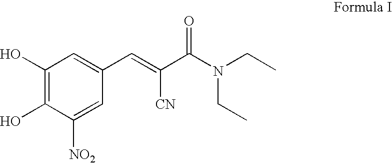
comprising
i) condensing 3,4-dihydroxy-5-nitro benzaldehyde with N,N-diethyl cyano acetamide in an organic solvent in the presence of an organic base and an acid under reflux conditions followed by distilling off the solvent under vacuum and treating the residue with a lower aliphatic carboxylic acid at 40-80° C.;
ii) extracting the residue with a chlorinated solvent and distilling off the solvent under vacuum;
iii) extracting the residue with an organic solvent to obtain crude N,N-diethyl-2-cyano-3-3,4-dihydroxy-5-nitrophenyl)acrylamide; and
iv) treating the crude product N,N-diethyl-2-cyano-3-(3,4-dihydroxy-5-nitrophenyl)acrylamide with a mixture of organic alcohol and organic acid in the molar ratio of the crude product to organic alcohol 1:5 to 15 and crude product to organic acid 1:1 to 3 under reflux conditions.

2. The process as claimed in claim 1, wherein the organic solvent used in step (i) is selected from lower aliphatic alcohol like methanol, ethanol, n-propanol, iso-propanol or mixtures thereof or is preferably ethanol.

3. The process as claimed in claim 1, wherein organic base used in step (i) is selected from piperidine, morpholine, N-methyl morpholine or pyrrolidine or is preferably piperidine and the acid used in step (i) is selected from lower aliphatic carboxylic acid like acetic acid or formic acid or inorganic acid selected from hydrochloric acid or sulphuric acid or is preferably acetic acid or formic acid.

4. The process as claimed in claim 1, wherein the residue in step (i) is treated 65° C.

5. The process as claimed in claim 1, wherein the chlorinated solvent used instep (ii) is selected from chloroform or methylene dichloride or is preferably methylene dichloride.

6. The process as claimed in claim 1, wherein the organic solvent used in step (iii) is selected from polar and non-polar solvents such as alcohols, ketones or esters or mixtures thereof or is preferably ester, preferably ethyl acetate.

7. The process as claimed in claim 1 wherein the mixture of organic alcohol and organic acid used in step (iv) is selected from aliphatic alcohol like methanol, ethanol, n-propanol or iso-propanol and lower aliphatic carboxylic acid like acetic acid, formic acid or trifluoro acetic acid or is preferably methanol and acetic acid.

8. The process as claimed in claim 1, wherein molar ratio of the crude product to alcohol is 1:8 and the crude product to organic acid is 1:2.

9. N,N-diethyl-2-cyano-3-)3,4-dihydroxy-5-nitrophenyl)acrylamide of the formula 1 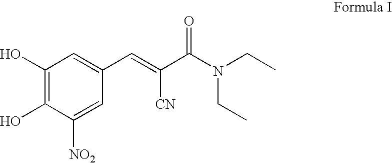
in high purity (>99.8%) and in good yield (53.68%) prepared by the process as claimed in claim 1.
